(19) United States
(12) Patent Application Publication
(10) Pub. No.: US 2006/0149602 A1
(43) Pub. Date: Jul. 6, 2006

(54) Methods and systems for online clinical trial screening

(75) Inventor: Gary Zammit, Norwalk, CT (US)

Correspondence Address: MANATT PHELPS AND PHILLIPS;ROBERT D. BECKER, 1001 PAGE MILL ROAD, BUILDING 2, PALO ALTO, CA, 94304, US

(73) Assignee: Clinilabs, Inc., New York, NY (US)

(21) Appl. No.: 11/298,168

(22) Filed: Dec. 8, 2005

Related U.S. Application Data
(63) Continuation-in-part of application No. 10/845,697, filed on May 14, 2004.
(60) Provisional application No. 60/470,953, filed on May 14, 2003.

(51) Int. Cl. A61B 5/00

(52) U.S. Cl. 705/3

(57) ABSTRACT

In a clinical trial program screening method, potential clinical trial candidates are driven to a screening website by, for example, banner ads, text messages, or other electronic advertisements. When a respondent clicks the advertisement, a multiple level screening method is conducted to screen qualified candidates for participation in a clinical trial program. Diagnostic, demographic and/or geographic data are collected from respondents and used to screen for predetermined clinical trial participation criteria. The effectiveness of the recruitment method is periodically evaluated and optimized to realize maximal recruitment efficiency.

### RELATED APPLICATIONS

This application is a continuation-in-part of application Ser. No. 10/845,697, filed May 14, 2004, which claims the benefit of provisional application Ser. No. 60/470,953, filed May 14, 2003, each of which is incorporated herein in its entirety by this reference.

### BACKGROUND

Technical field: The present invention relates, in general, to online data collection and processing and, more specifically, to program participant recruitment and screening through networks.

Related art: Clinical trial programs are essential in the advancement of medicine and health care. They provide heath care researchers and professionals with valuable information about the efficacy and possible adverse effects of various treatments. They also provide patients and their physicians with access to new and alternative treatments.

With the widespread use of the Internet, patients and their physicians increasingly rely on the Internet in seeking information about heath care, disease prevention and treatment. Health care providers and researchers would like to take advantage of the Internet in recruiting and screening potential participants for clinical trial programs. For example, about 92% of physicians in the United States and about 80% of physicians in Europe who responded to a survey are using or have interests in using the Internet to find information about clinical trials for their patients. Another survey indicates that more than 68% of adults in the United States have sought health related information on the Internet. In addition, more than 40% of clinical trial program participants indicate that they have used online resources to obtain infonnation about research programs.

Accordingly, it would be advantageous to provide a method for online recruitment and screening of potential participants for a clinical trial program. It would also be advantageous for the method to be able to reach targeted audiences through the Internet. It would also be advantageous for such a method to be able to screen and enroll the potential participants efficiently via computer networks such as the Internet. It would be of further advantage to be able to securely protect the privacy of the potential participants.

### BRIEF SUMMARY

One embodiment provides a method for online data collection and processing, including clinical trial subject recruitment and/or screening, via networks such as the Internet. The method may be incorporated in software for causing a computer to perform the method. In accordance with one embodiment, a method for recruiting and/or screening participants for a clinical trial program includes online banner advertisements strategically targeted to audiences according to their demographic profiles and/or geographic profiles. In accordance with another embodiment, the method may also include recruiting and/or screening participants via text advertising, keyword search terms from web search engines, and other forms of advertisement or media relations techniques used to attract Internet users.

In accordance with another embodiment, photographic images or icons are used to guide the respondents through different levels of an automated screening method. An automated progressive screening method is employed, which requires the respondents to pass several levels of screening in order to qualify for participation in a clinical trial program.

In accordance with another embodiment, automated e-mail messages are generated from the respondent to the organization that conducts the recruiting program, indicating the respondents' interest and qualification in participating in the program. In one embodiment, the respondent's interest and qualification information is posted on a secure, interactive website or database accessible by clinical trial sites used to identify potential study participates. The website includes the respondent's information and contact information so that the clinical trial site can contact individual respondents and update the respondent's profile as needed through the length of clinical trials program.

In accordance with another embodiment, the data generated by the recruiting method is securely stored in a data storage unit within the organization. The data is not shared with sources outside of the organization to maintain the confidentiality and readiness for auditing.

In accordance with another embodiment, the recruiting method periodically evaluates the effectiveness of the advertisement and modifies the advertising strategy to maximize the recruitment efficiency.

### BRIEF DESCRIPTION OF THE DRAWINGS

In the several figures of the drawings, which illustrate exemplary embodiments of the invention:

FIG. 1 illustrates an online clinical trial recruiting and/or screening method;

FIG. 2 illustrates another online clinical trial recruiting and/or screening method;

FIG. 3 illustrates yet another online clinical trial recruiting and/or screening method;

FIG. 4 is a table setting forth exemplary therapeutic areas for which clinical trials may be conducted;

FIG. 5 illustrates a first screening level;

FIG. 6 illustrates a second screening level;

FIG. 7 illustrates a third screening level; and

FIG. 8 illustrates a fourth screening level.

### DETAILED DESCRIPTION OF THE PREFERRED EMBODIMENTS

Various embodiments are described hereinafter with reference to the drawings. Elements of like structures or function are represented with like reference numerals throughout the drawings. The drawings are only intended to facilitate the description of specific embodiments of the invention. They are not intended as an exhaustive description of the invention or as a limitation on the scope of the invention. In addition, an aspect described in conjunction with a particular embodiment is not necessarily limited to that embodiment and can be practiced in conjunction with any other embodiments.

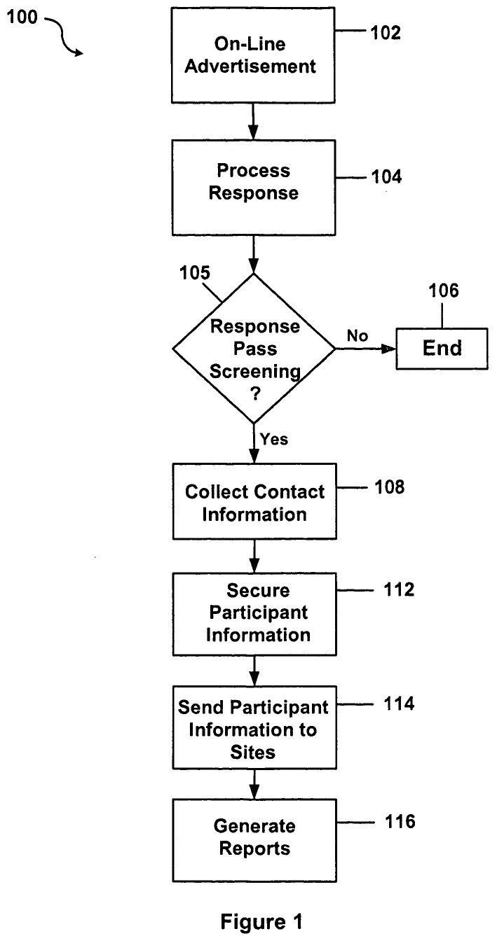

FIG. 1 illustrates an online clinical trial recruiting method 100. By way of example, method 100 is directed to recruitment of participants in a clinical trial program through the Internet. However, the Internet is merely an example of a suitable computer network. Method 100 is applicable in applications that include soliciting and processing responses through other types of computer networks, such as, for example, Intranets, Local Area Networks (LANs), Wide Area Networks (WANs), and the like.

In step 102, advertisements about the clinical trial program are placed on various websites. Preferably, the advertisements include banners, icons, and/or other images depicting one or more specific disorders for which the clinical trial program is conducted. For example, if the clinical trial program concerns a study in the area of depression, the advertisement may include an image that depicts a person who appears depressed. The advertisement may also include a logo of the organization that is conducting the program or recruiting for participation in a clinical trial program.

The advertisements for the clinical trial program may be strategically positioned on websites that target specific demographic profiles. For example, if the clinical trial program is for the study of Alzheimer's disease, the advertisements may be placed on websites that seniors frequently visit. The advertisement may also be placed on websites that appear in specific geographic locations if the disorder under study has certain geographic profiles or concentration and/or the recruitment seeks to reach certain basis of geography based upon the geographic recruitment goals of the clinical trial program.

When a potential participant or respondent viewing the advertisement clicks a link in the advertisement to access web page, a screening page is opened in step 104. The web page may be a page on a website that hosts the advertisement. Alternatively, the web page is a page on the website operated by an organization that conducts the clinical trial program recruitment. Step 104 includes questions to be answered by the respondents, which may include health related questions. Based on the responses, a determination is made at step 105 regarding whether the respondent qualifies to be a participant for the clinical trial program.

If the responses indicate that the respondent is not eligible to participate in the clinical trial, the respondent is so informed and the method 100 is terminated in step 106 and a notice may be displayed suggesting that the respondent consult his/her primary care physician regarding the health condition. In addition, step 106 may display a query to the respondent asking whether he/she wants his/her information entered into a database for consideration in future clinical trial programs.

If the responses suggest that the respondent is eligible to participate in the clinical trial, method 100, in a step 108, informs the respondent about his/her eligibility to the clinical trial program. In addition, at step 108 contact information is collected, e.g., respondent's home address, zip code, telephone number, e-mail address, the name and address of the respondent's primary care physician, and so on, from the respondent. From a respondent's address information, eligible respondents are referred to clinical trial sites in their geographic area. The appropriate clinical trial site may then contact the respondent by phone to perform additional screens to ultimately determine the respondent's eligibility to participate in a trial. In one embodiment, method 100 suggests that the respondent seek advice from his/her primary care physician in response to the respondent refusing or otherwise failing to provide the contact information.

In step 112, method 100 transmits and securely stores the responses and contact information of eligible respondents on a data storage unit operated by the organization that conducts the clinical trial program recruitment. In one embodiment, steps 104, 105, and 108 of method 100 are performed on an outside website other than the website operated by the organization. In this embodiment, in step 114, an e-mail including the data about the respondent is sent to the organization website, where it is securely stored. In addition, all data about the respondent are deleted from the outside website. In another embodiment, steps 104, 105, and 108 of method 100 are performed on a secure organization operated website. In this embodiment, the data is transmitted to the data storage unit for secure storage. In any of the above embodiments, in step 116, reports may be generated based on the respondents' data. These reports can be generated via an interactive website which creates individualized reports requested by investigators and clinical trial sponsors.

Reports may be posted to a secure, interactive website that can be accessed by clinical investigators' sites or by the clinical trial sponsors. If an investigator goes to the website, he can see only his data. If a sponsor goes to the website, it can see data from all sites participating in its trial. The reports show a variety of data including, but not limited to participant info and the status of the participants (“awaiting contact,” “contacted,” “seen in office,” “enrolled in trial”). The investigators or sponsors can interact with the report module to enter information or comments regarding the participants. For example, after a referred participant is screened in the office, the investigator's site can change the participant's status from “contacted” to “screened.” Other reports can also be generated that provide relevant information to investigators and sponsors including, but not limited to: (a) participant characteristics (e.g., if participants with cancer are sought, relevant data may be forwarded to the investigators and sponsors including how long the average person has had cancer, or how many of respondents report cancer pain, etc.), (b) the location of participants (e.g., in what zip codes do most cancer patients reside), and (c) the overall effectiveness of the ad campaigns.

In accordance with some embodiments, the storage and management of the data generated in method 100 is secured to provide the privacy of the respondents. In one embodiment, the data is managed in accordance with the Health Insurance Portability and Accountability Act (HIPAA) and the confidentiality, security, and audit requirements of 21 CFR Part 11 or other applicable laws and regulations.

The effectiveness of the advertisement may be analyzed or evaluated periodically, e.g., daily, weekly, monthly, and so on. In one embodiment, the numbers of clicks generated at various steps in method 100 is evaluated for each advertisement. The evaluation is used for modifying the advertising strategy or format to achieve optimum efficiency and maximize program participant recruitment.

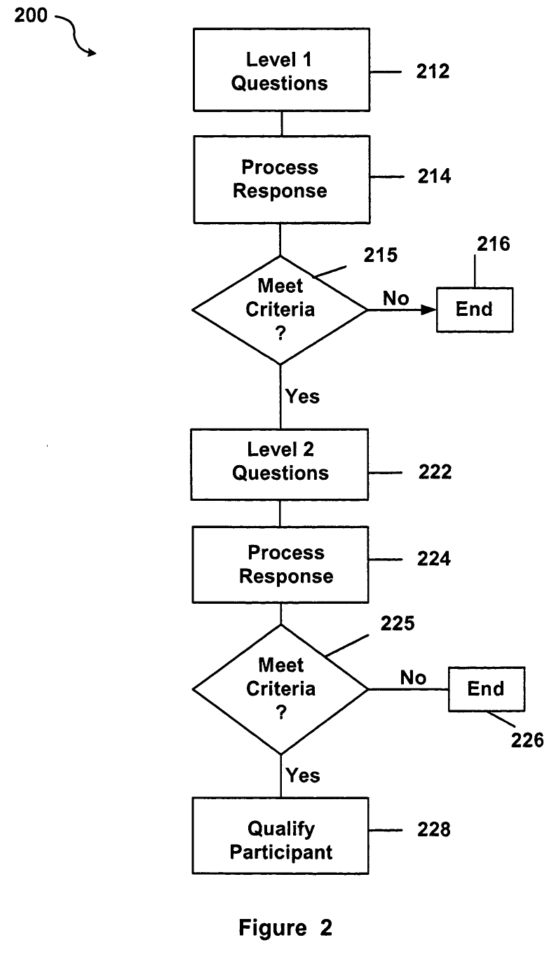

FIG. 2 illustrates an online screening method 200. By way of example, method 200 is described herein as performing screening steps 104 and 105 of method 100 illustrated in FIG. 1. Method 200 is preferably a multiple level screening method.

During the first level of screening, a respondent who clicks on the advertisement banner, image, or icon is directed to a landing site that presents a short list of symptoms that are characteristic of the disorder in which the respondent has expressed interests (step 212). For example, the respondent may be presented with the following: “Do you suffer from mood swings? Have you lost interest in your usual activities? Have you lost enjoyment for things that you usually find pleasurable?” A photographic image or icon depicting a specific disorder may also be displayed.

In step 214, the respondent's responses to the short list of questions are processed. If the responses do not meet a predetermined criterion (step 215), the screening method terminates in step 216 at the first level of screening. If the responses meet the predetermined criterion, method 200 proceeds to the second level of screening. In other words, the respondent who passes the first level of screening is directed to the second level of screening.

At the second level of screening, the respondent is presented with a questionnaire (step 222) that is divided into a plurality of sections. Each section includes one or more questions. In a preferred embodiment, the questionnaire may includes three to five sections and each section may include approximately five questions. However, the number of sections and the number of questions in each section can be adjusted for different screening processes. Preferably, the questionnaire is not too long for the average attention span of the target audience. Successful completion of each section enables the respondent to proceed to the next section.

In step 224, the respondent's responses to the questionnaire are processed. If the responses do not meet a predetermined criterion (step 225), the screening method terminates in step 226. If the responses meet the predetermined criterion, method 200 proceeds to qualify the respondent for participation in a clinical trial program (step 228). In accordance with one embodiment of the present invention, the respondent is directed to step 108 of method 100 shown in FIG. 1 for collection of contact information.

It should be understood that method 200 is not limited to including two screening levels. In accordance with alternative embodiments of the present invention, screening method 200 may include any plural number of screening levels, e.g., three, four, five, and so on. Successful completion of one level enables the respondent to proceed to the next level. It should also be understood that screening method 200 is not limited to serving as steps 104 and 105 in program participant recruiting method 100 illustrated in FIG. 1. In accordance with the present invention, screening method 200 can be used for any interactive data processing method.

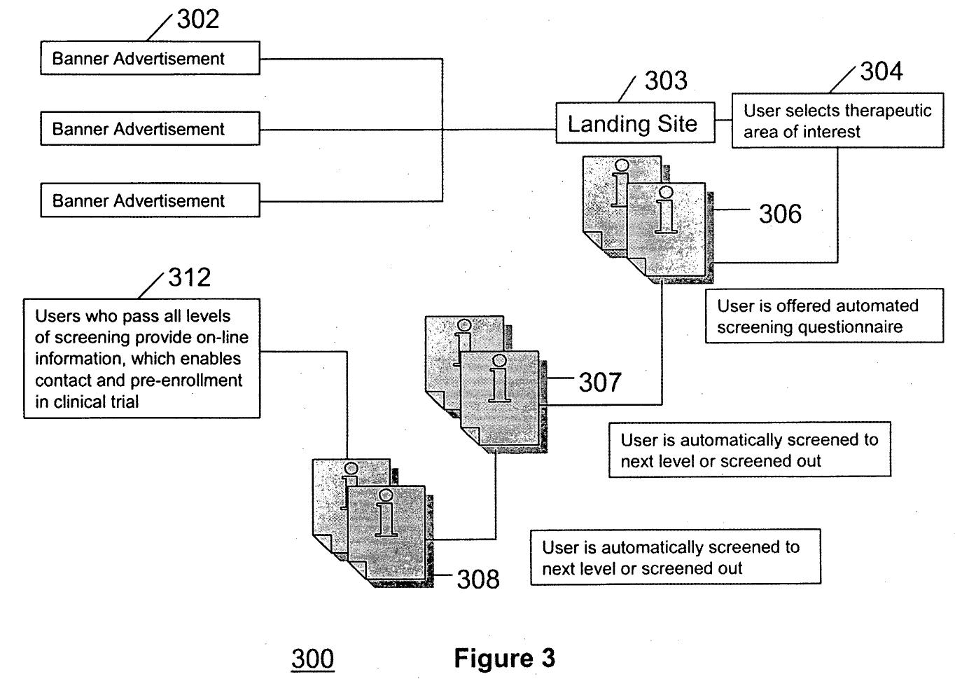

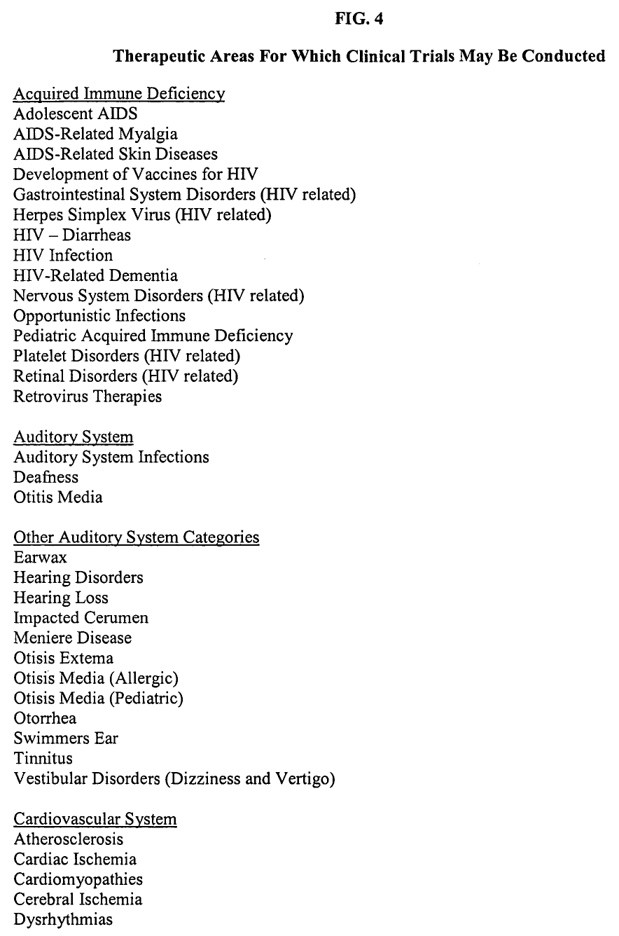

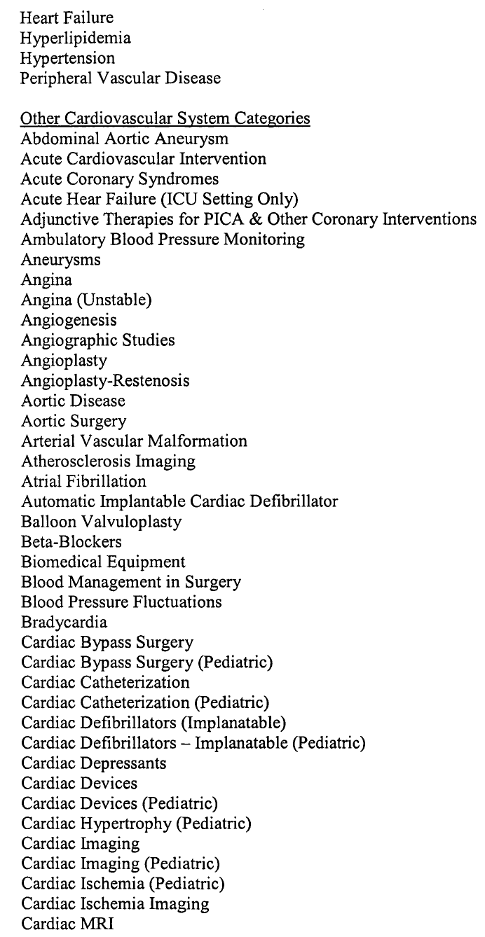

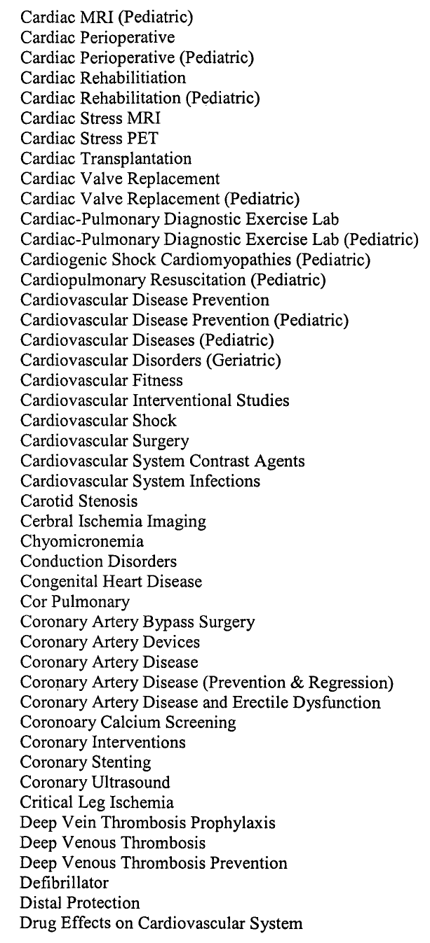

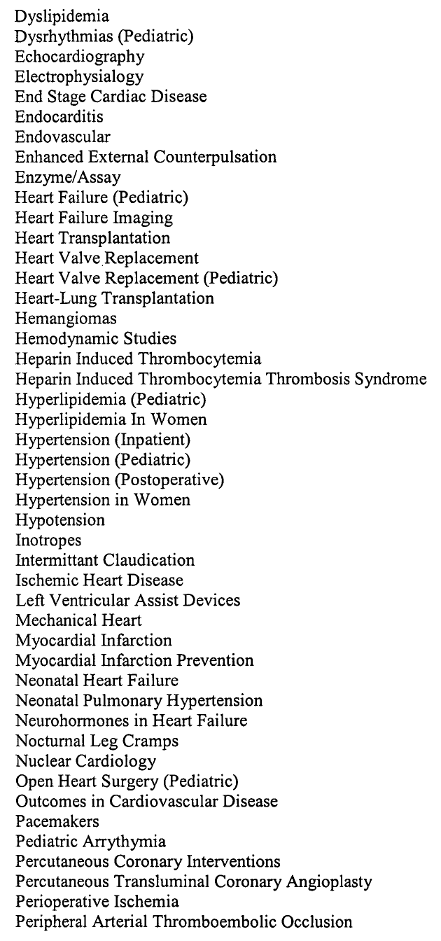

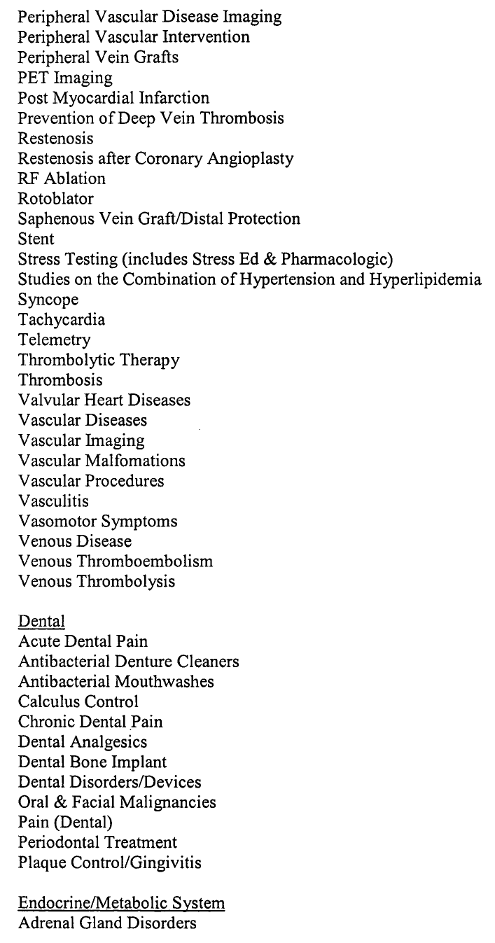

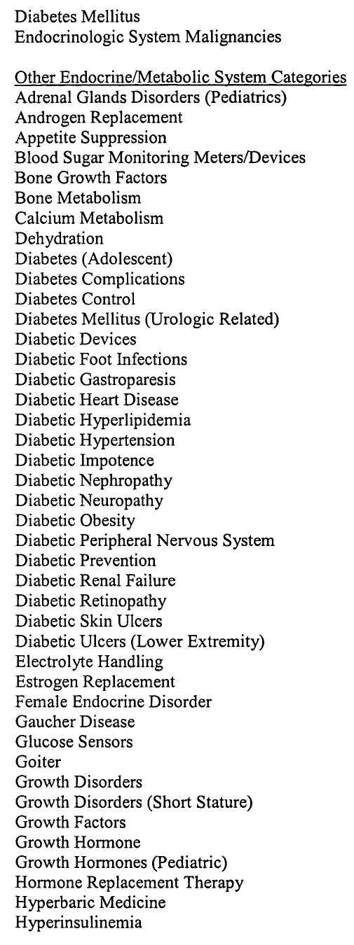

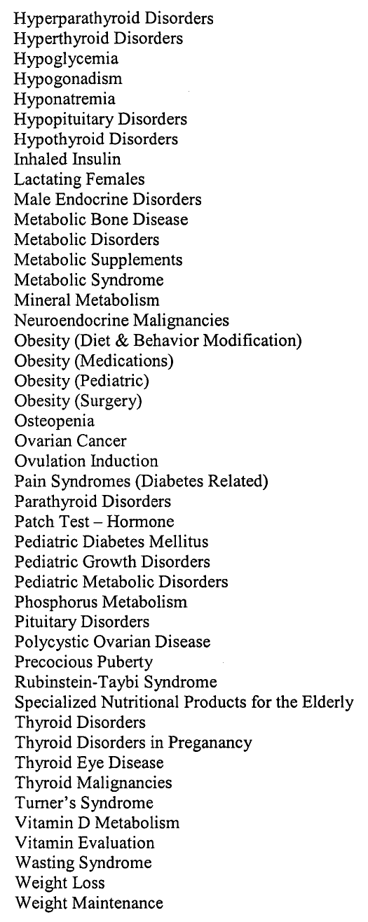

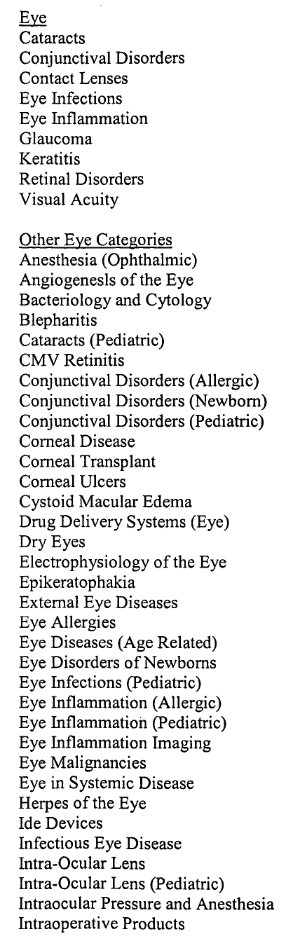

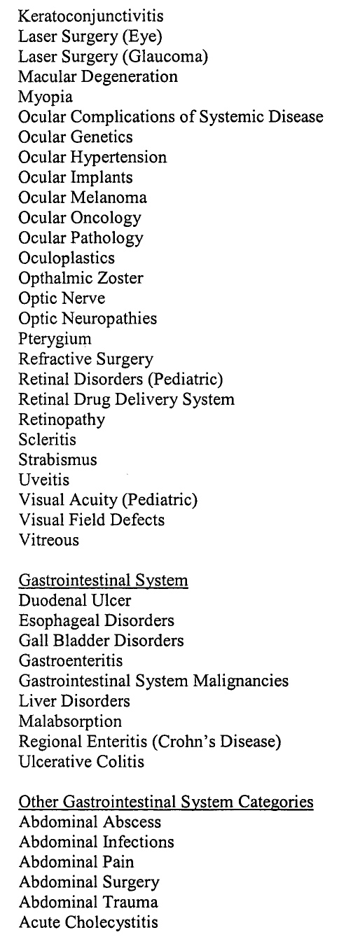

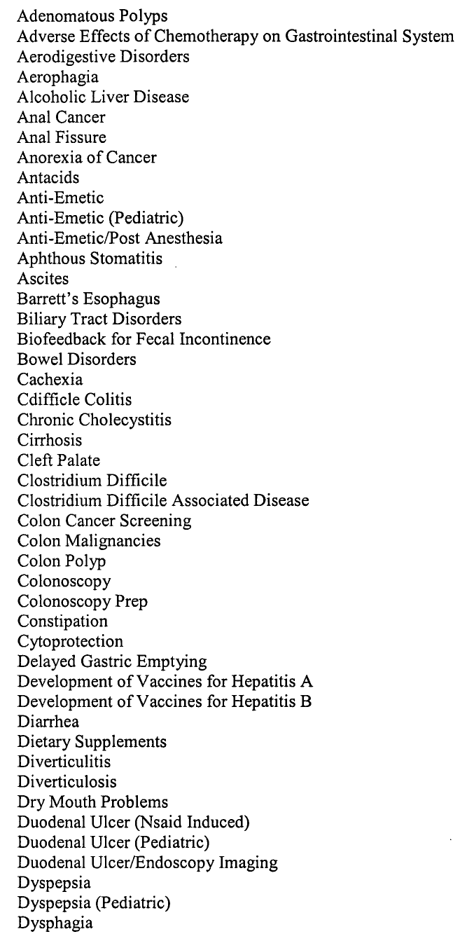

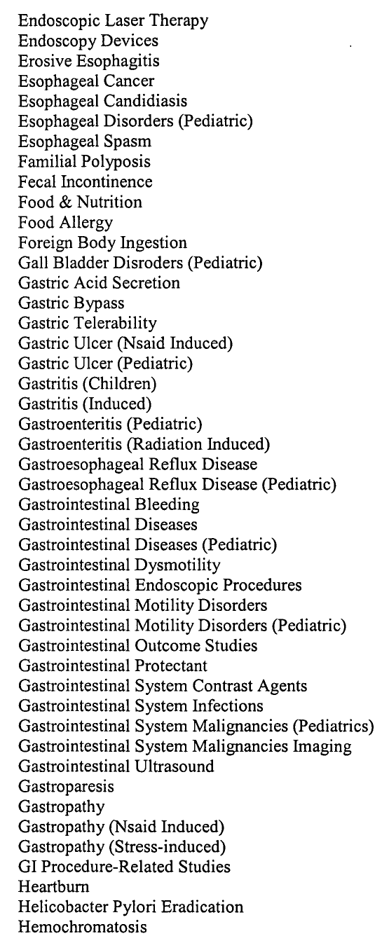

FIG. 3 illustrates an online clinical trial recruiting method 300 in accordance with the present invention. By way of example, method 300 recruits participants in a clinical trial program through the Internet. However, this is not intended as a limitation on the scope of the present invention. Method 300 is applicable in applications that include soliciting and processing responses through any network.

In step 302, advertisements about the clinical trial program are placed on various websites. Preferably, the advertisements include banners, icons, and/or other images depicting a specific disorder, for which the clinical trial program is conducted. The advertisement may also include logos of the organizations that are conducting the program or recruiting for participation in a clinical trial program. In accordance with an embodiment of the present invention, the advertisements for the clinical trial program are strategically positioned on websites that target specific demographic profiles. The advertisement may appear in specific geographic locations if the disorder under study has a geographic profile or the clinical trial program has a geographic recruitment goal.

When a potential participant or user viewing the advertisement clicks a link in the advertisement, the user is directed to a landing site (step 303) that conducts the screening method of the potential participants for participation in a clinical trial program. The landing site may be located on a website that hosts the advertisement. Alternatively, the landing site is located on a network server operated by an organization that conducts the clinical trial program recruitment.

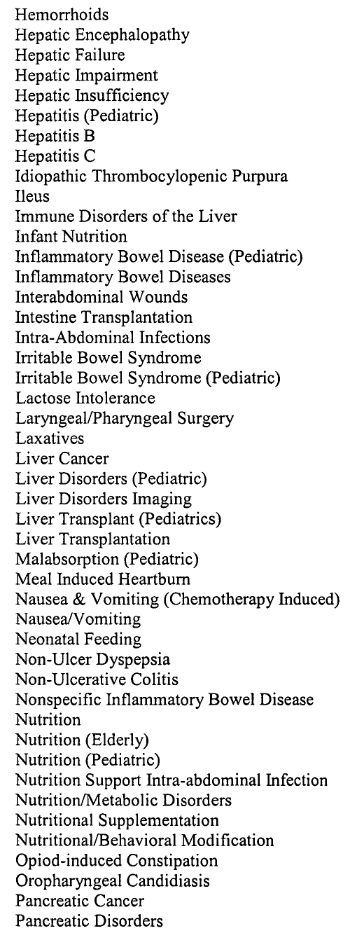

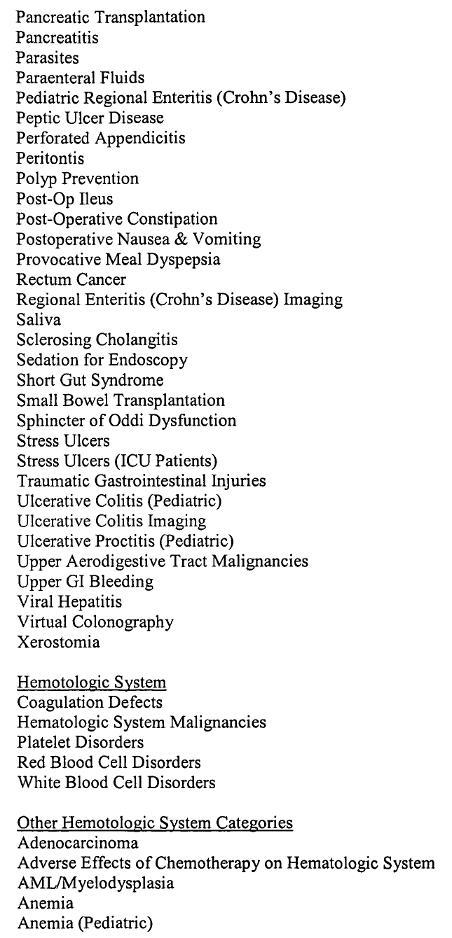

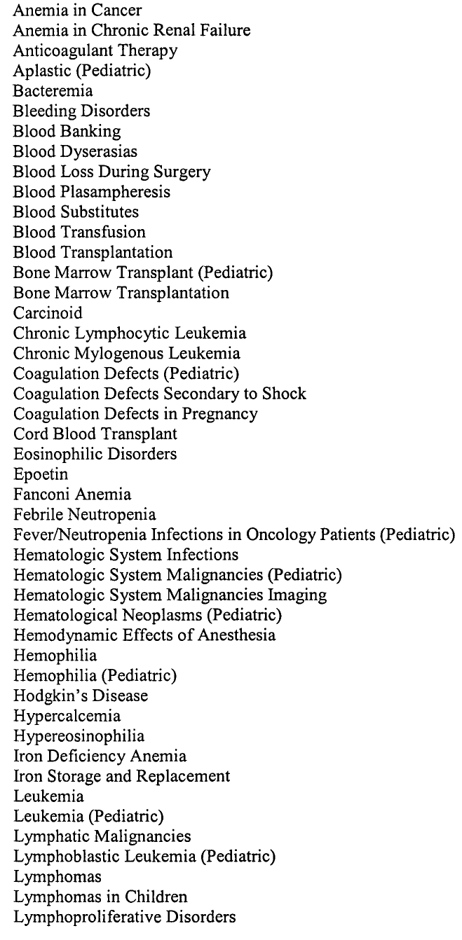

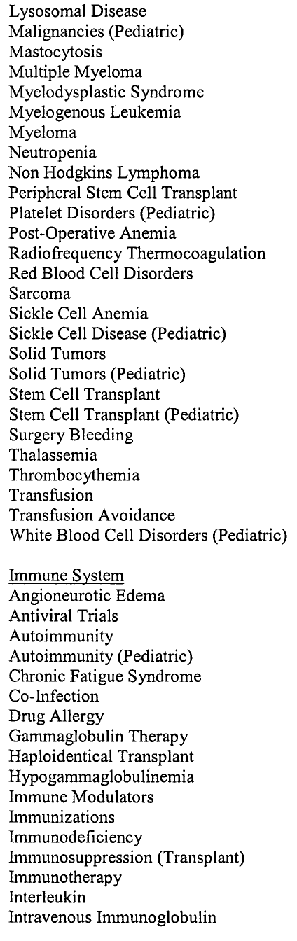

In step 304, the user selects a therapeutic area of interest, e.g., treatment for depression, insomnia, bipolar disorder, cancer, high blood pressure, and so on. The table provided in FIG. 4 sets forth exemplary therapeutic areas for which clinical trials may be conducted. This broadly includes various clinical areas of study, and is more specifically enumerated in the table in FIG. 4 under the following categories: acquired immune deficiency, auditory system, cardiovascular system, dental, endocrine/metabolic system, eye, gastrointestinal system, hemotologic system, immune system, infectious diseases, mental disorders, musculoskeletal system, nervous system, pharmacology, reproductive system, respiratory system, skin & soft tissue, and the urinary system.

Based on the user selection, method 300, in step 306, presents the user with a predetermined screening questionnaire, which includes one or more questions related to the user selected therapeutic area of interest. The user's response to the questionnaire may disqualify the user from the clinical trial program, in which case method 300 informs the user and preferably suggests that user seek advice from his/her primary care physician.

If the user passes the questionnaire presented in step 306, method 300 directs the user to the next level of screening (step 307). Optionally, method 300 includes a third level of screening (step 308) for the user in response to the user passing the second level of screening. It should be understood that method 300 may include additional levels of screening not shown in FIG. 3.

If the user is determined to be eligible to participate in the clinical trial by passing all levels of screening, method 300, in a step 312, informs the user about his/her eligibility to the clinical trial program. In addition, method 300 collects contact information data, e.g., user's initials, telephone number, e-mail address, zip code, and so on, from the user. This data enables the organization to contact the user and enroll the user into the clinical trial program that the user selects in step 304. In accordance with one embodiment, method 300 suggests that user seek advice from his/her primary care physician in response to the user refusing or otherwise failing to provide the contact information.

Preferably, method 300 transmits and securely stores the responses and contact information data of eligible users on a data storage unit operated by the organization that conducts the clinical trial program recruitment. In one embodiment, an e-mail or other message including the data about the user is sent to the organization, where it is securely stored and otherwise properly handled consistent with applicable laws and regulations. In another embodiment, the user's responses, contact information and data are accessible on a secure, interactive website.

In accordance with some embodiments, the storage and management of the data generated in method 300 is secured to provide the privacy of the users. In one embodiment, the data is managed in accordance with the confidentiality, security, and audit requirements of HIPAA and 21 CFR Part 11.

In accordance with other embodiments, the effectiveness of the advertisement is periodically analyzed or evaluated. The numbers of clicks generated at various steps in method 300 is evaluated for each advertisement. The evaluation is used for modifying the advertising strategy or format to achieve optimum efficiency and maximize program participant recruitment.

The method may be implemented by software, accessible alone or as part of a network site, e.g., a website, designed for the recruitment of subjects into clinical trials. Such embodiments may be implemented by a system having a server linked to a network, such as a LAN or the Internet, such that the invention is accessible to users via the network, e.g., to Internet users via a website. According to some embodiments, use is made of a series of automated screening forms, queries, and responses that are designed to identify appropriate clinical trial candidates. The exemplary embodiment described below comprises a four-level screening method according to the invention.

In this exemplary embodiment, network users are driven to a recruitment site by text, banner, or other forms of Internet or traditional advertisements, including, for example, a keyword or other search result provided by a search engine or other search facility. Contemporary, widely-used search engines include Google®, Yahoo!®, AltaVista® and the like. Users may enter the recruitment site via any other sources, such as links from other websites or as a result of random visits.

Screening Level 1

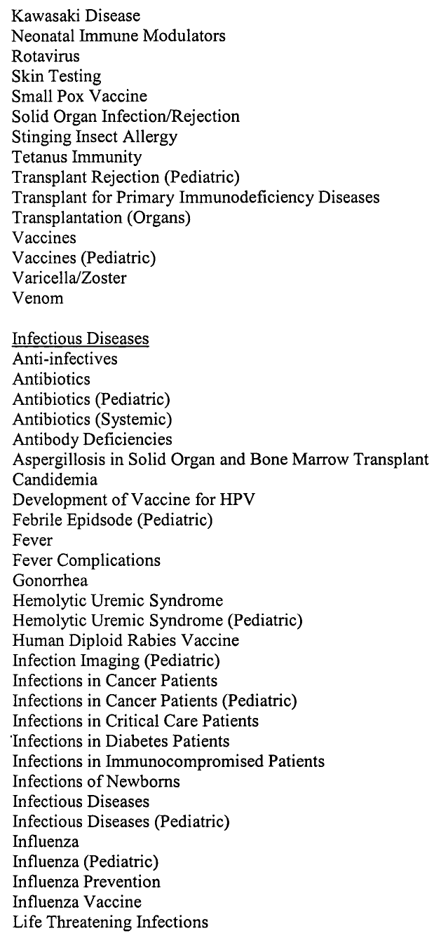

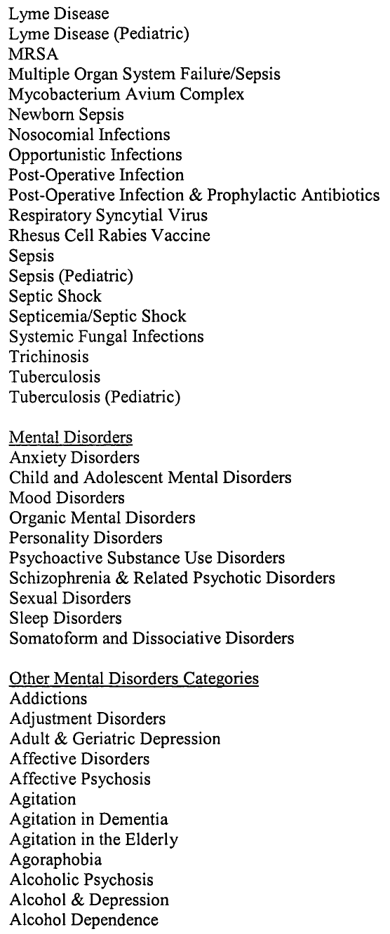

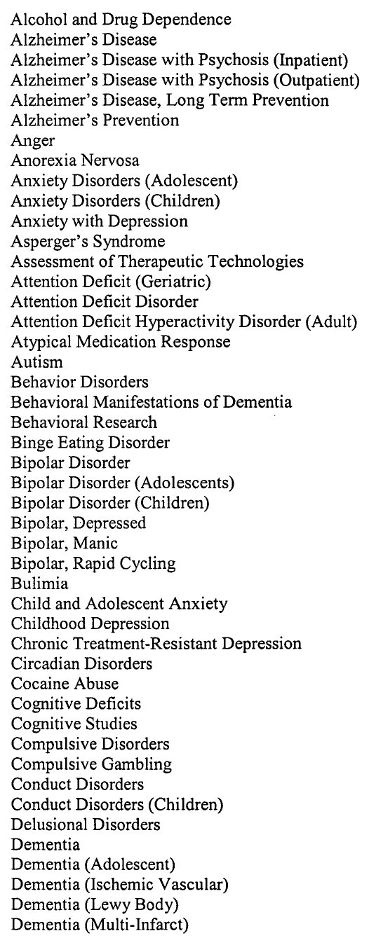

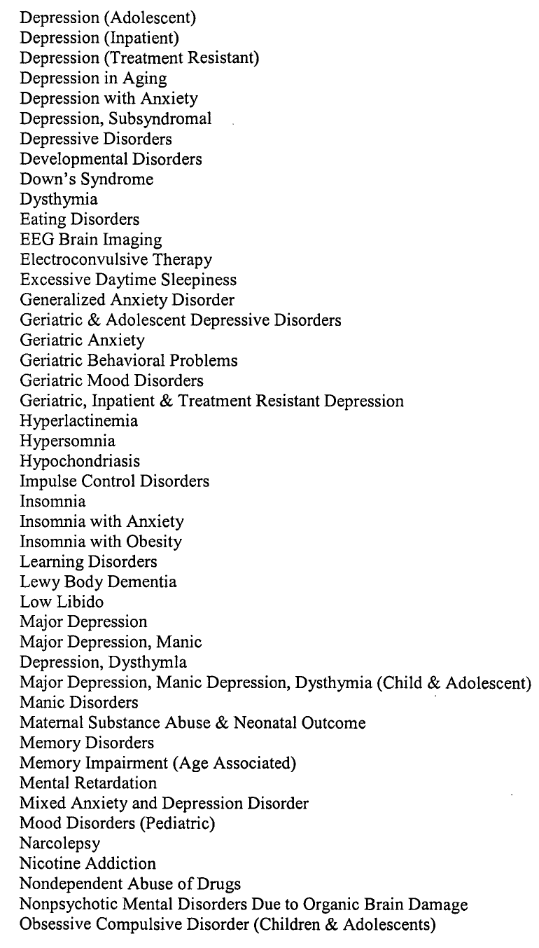

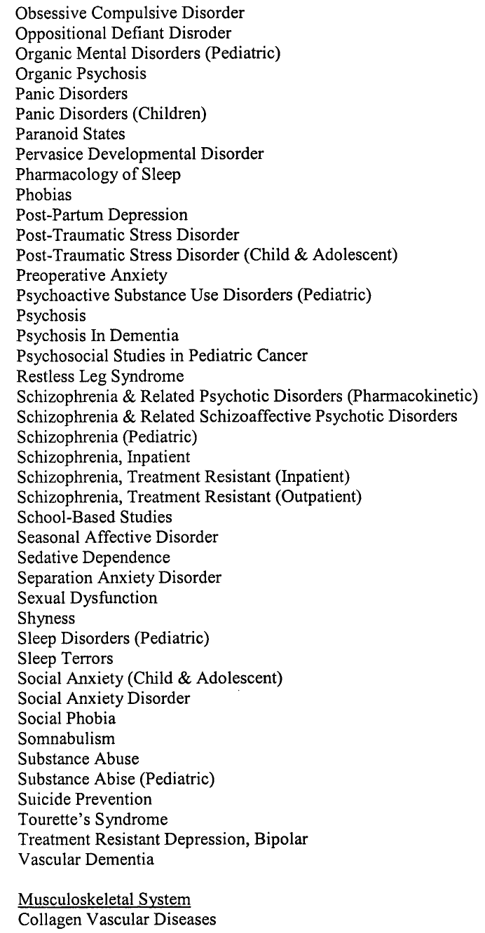

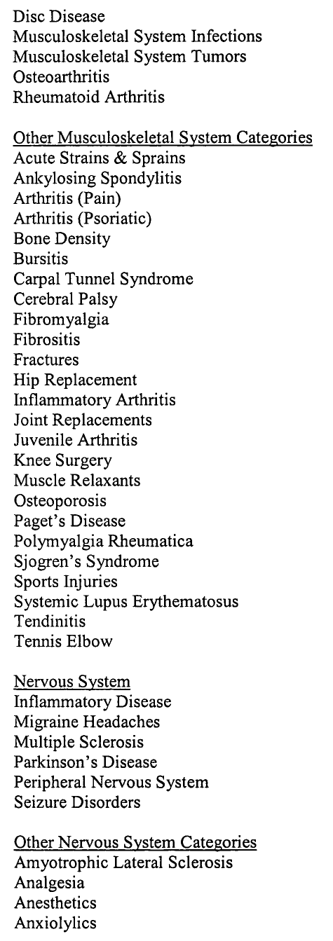

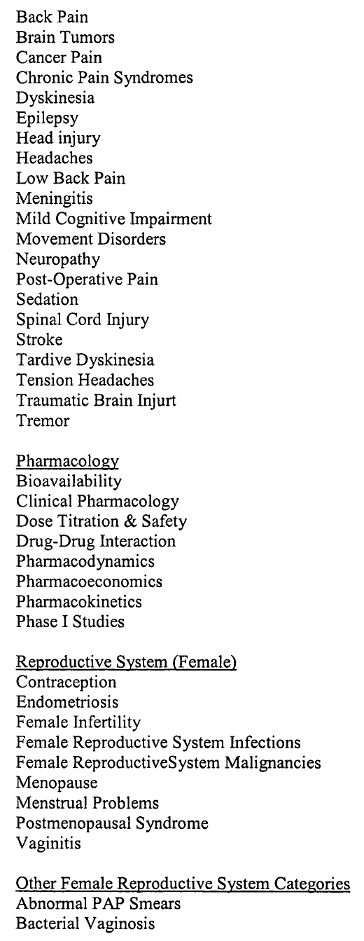

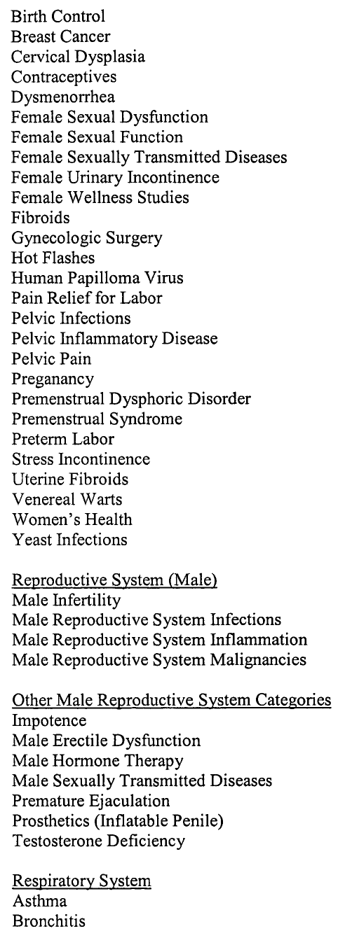

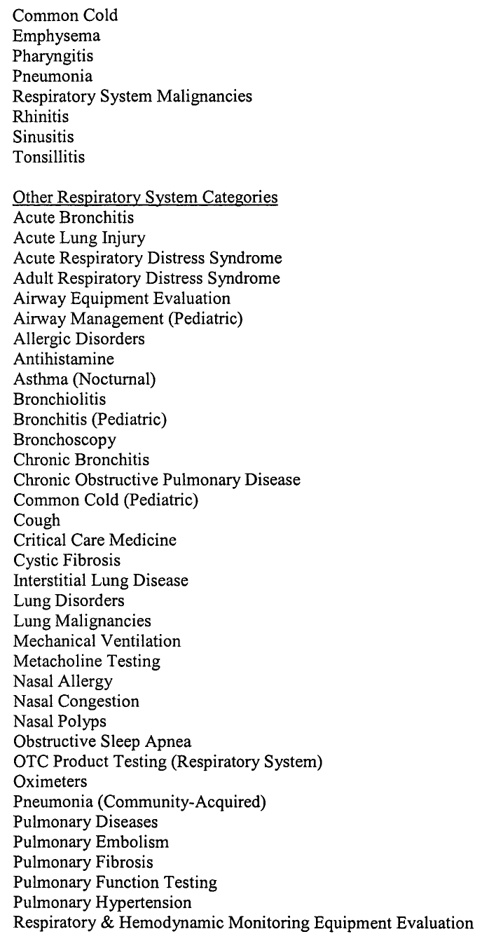

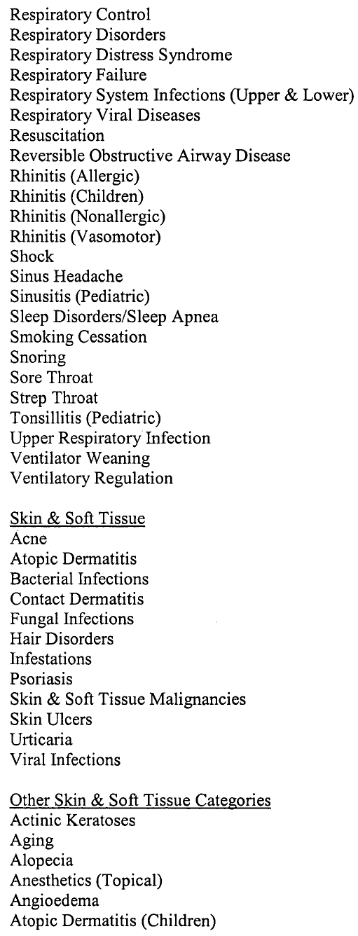

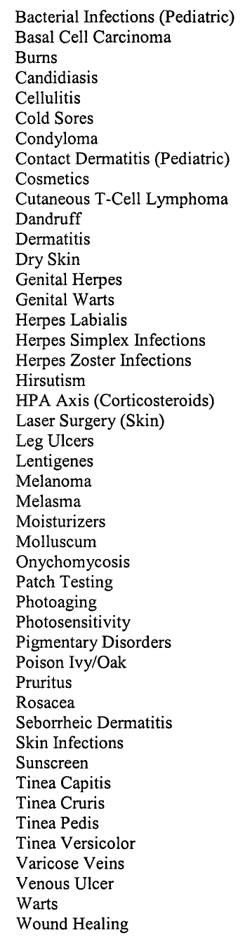

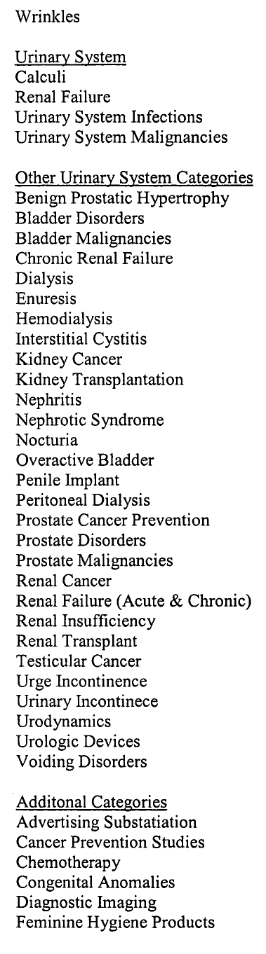

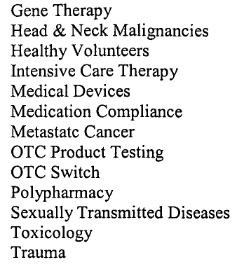

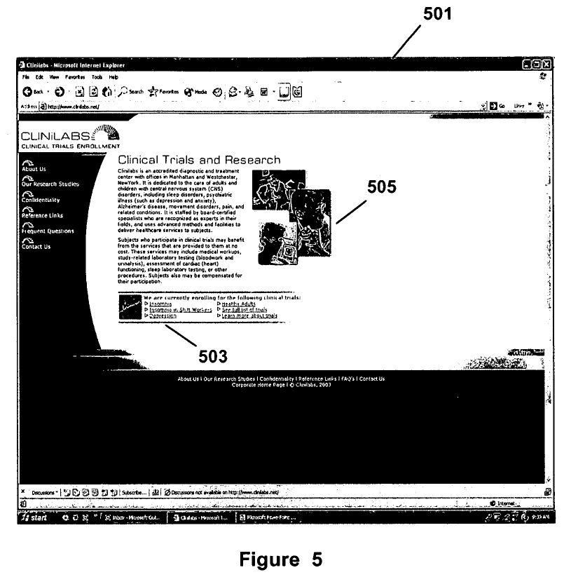

As shown in FIG. 5, upon visiting the recruitment website, Internet users view a home page 501 that provides general information, as well as specific information that identifies specific therapeutic areas of interest or specific clinical trials that might be of interest to the user. Specific therapeutic areas of interest or trials may be identified by text 503, graphics, or photo images 505, or some combination thereof. For example, if a user is interested in clinical trials related to insomnia, he/she may select “insomnia” as a therapeutic area of interest by pointing his/her computer mouse indicator to either the text 503 related to insomnia, or the photo image 505 of a person representing this disorder and “clicking” to select. Clicking on this item represents the first level of automated screening performed by this embodiment of the invention, referred to herein as Screening Level 1.

Screening Level 2

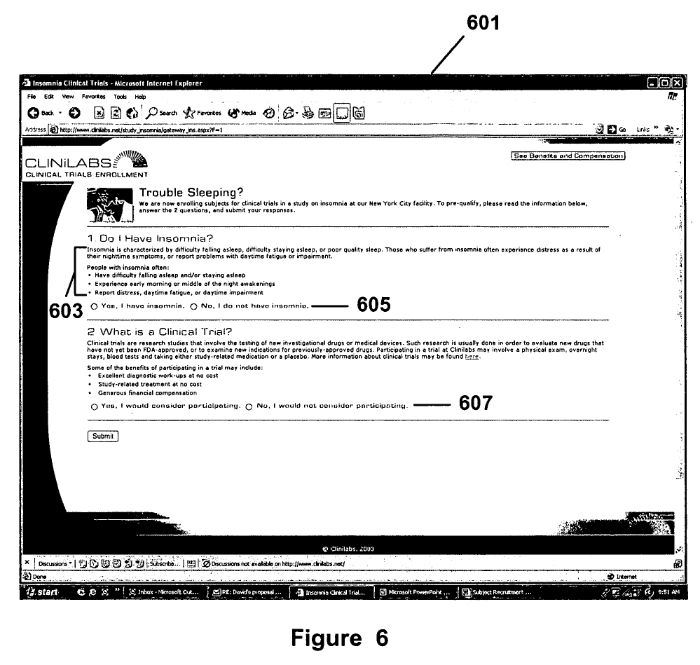

As shown in FIG. 6, upon clicking on a therapeutic area, the user is taken to a landing page 601, which represents the second level of screening (Screening Level 2). The user is exposed to information 603 and an initial screening question 605 (or series of initial screening questions) regarding the specific disorder or therapeutic area of interest. The user is asked to respond to the question(s) in order to determine if the user has symptoms consistent with the disorder. These questions require a personalized response. For example, the information states “people with insomnia often report . . . ” and then the user is asked to indicate “Yes, I have insomnia,” or “No, I do not have insomnia.”

At this level of screening the user also is given information about clinical trials and an initial screening question 607 (or series of initial screening questions) regarding the user's interest in participation in a clinical trial. For example, the user may be asked to indicate “Yes, I would like to participate in a clinical trial,” or “No, I do not wish to participate.”

Screening Level 3

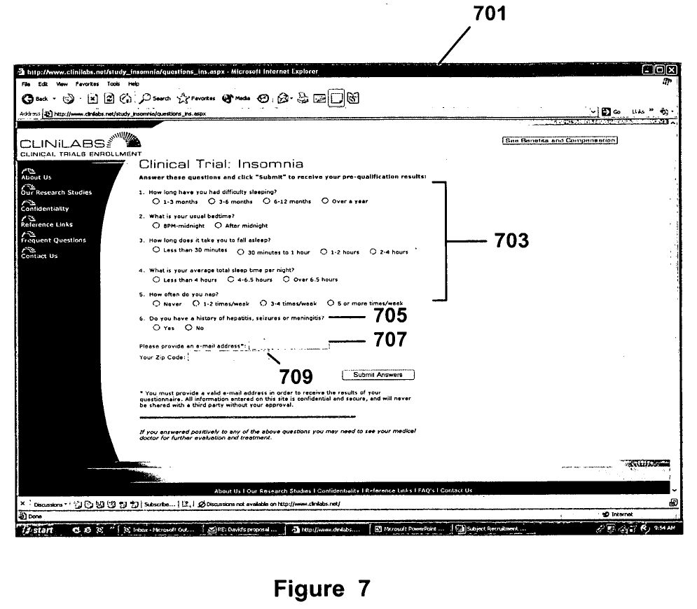

As shown in FIG. 7, users who respond affirmatively to screening questions posed at Screening Level 2 (FIG. 6) are directed to a disorder-specific screening questionnaire 701, referred to herein as Screening Level 3. Each questionnaire is designed to determine if the user meets general diagnostic criteria 703 for a specific disorder, and general inclusion/exclusion criteria 705 commonly used in clinical trials. The questionnaire also asks the user for basic contact information including, for instance, (a) name, (b) e-mail address 707, (c) telephone number, and/or (d) zip code 709. The user is asked to complete the questionnaire and submit his/her information.

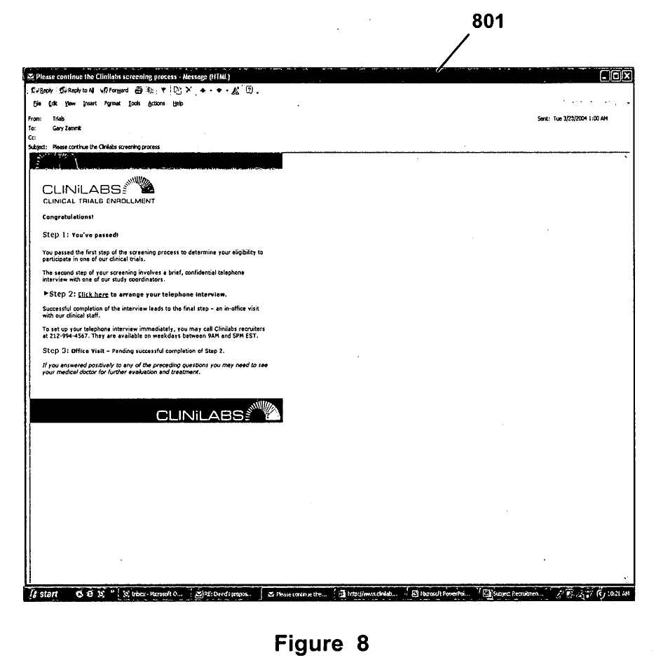

As shown in FIG. 8., users who respond to the disorder-specific screening questionnaire in a manner that indicates that they have a disorder, without exclusionary characteristics, are given an immediate message 801 that informs them that they have “passed” the initial screening. The immediate message 801 may include additional information regarding clinical trials and any further screening that may be required.

Users who respond negatively to one or both the screening questions posed at Screening Level 2 are notified that they have “not passed” the pre-screening. They also may be given other information about clinical trials, health, or other topics.

Screening Level 4

Users who pass Screening Level 3 receive an automated e-mail at a pre-determined interval, e.g., 24-hours after they have submitted their Screening Level 3 responses. This e-mail prompts users to re-confirm their interest in participating in a clinical trial. Users who respond affirmatively pass Screening Level 4.

Files containing demographic and health information from all users who pass Screening Level 4 are sorted by identifiers. A variety of identifiers may be used to sort the data. For example, zip code is one identifier that can be used to sort data.

Sorted data are filed, or may be sent to sponsors or sites involved in clinical trials. For example, a file from a patient who passed Screening Level 3 for insomnia who reported that he resides in zip code “10025” might be sent to a clinical trials site in that zip code. The clinical trial site may then contact the patient regarding, for example, enrollment in the clinical trial or further screening.

Data obtained from users, such as demographic data, geographic data and any other data, may be mined for information of educational, research or commercial value. Such data mining may be useful, for example, in the design of clinical trials and in the selection of sites for clinical trials.

In exemplary embodiments, users who successfully complete all levels of screening are contacted by clinical trial staff for a telephone interview, at which time they may be screened further to determine if they meet study-specific inclusion/exclusion criteria.

Guided Search

Users' searches may be guided using automated features. For example, if a person interested in the “insomnia” therapeutic area indicates that he/she is elderly, the application might guide the patient to complete screening items geared for elderly insomnia.

Compliance

Some embodiments of the invention include features that are consistent with compliance with state, federal and other laws and regulations that govern the collection, handling and use of electronic data in clinical research, including HIPAA and 21 CFR Part 11.

Reports

Some embodiments of the invention may manually or automatically generate activity reports which can be used to provide summaries of user demographics, symptom presentation, geographic data, performance statistics, or to generate invoices.

While the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention.

What is claimed is:

1. A method for screening clinical trial candidates, comprising the steps of:
providing a first level screening module at a network location, the first level screening module comprising at least one selectable element regarding at least one clinical trial program relating to at least one medical condition;
if a candidate selects the selectable element, then directing the candidate to a second level screening module, the second level screening module comprising informative matter regarding the clinical trial program and regarding the medical condition and enabling the candidate to submit input regarding interest in participating in the clinical trial program;
if the candidate submits input indicating interest in participating in the clinical trial program, then directing the candidate to a third level screening module, the third level screening module comprising a questionnaire for obtaining information from the candidate regarding diagnostic criteria for the medical condition and inclusion criteria for the clinical trial program; and
if the candidate submits information via the questionnaire that meets predetermined diagnostic and inclusion criteria, then providing an indication to the candidate that initial screening for the clinical trial program has been successful and generating an automated communication to the candidate requesting confirmation of the candidate's interest in participating in the clinical trial program.

2. The method according to claim 1, further comprising, if the candidate submits information via the questionnaire that meets predetermined diagnostic and inclusion criteria, generating an automated communication to a clinical trial program administrator.

3. The method according to claim 2, wherein generating the automated communication to the clinical trial program administrator comprises generating an automated communication including at least some information obtained from the candidate via the questionnaire regarding diagnostic criteria for the medical condition and inclusion criteria for the clinical trial program.

4. The method according to claim 1, wherein the network location comprises an Internet site or an Intranet site.

5. The method according to claim 1, wherein the selectable element comprises the name of the medical condition or a graphic representation of the medical condition.

6. The method according to claim 1, further comprising maintaining a secure database of information obtained from the candidate and information regarding the status of screening the candidate for the clinical trial program.

7. The method according to claim 1, wherein the questionnaire includes one or more queries for obtaining contact information for the candidate.

8. The method according to claim 7, wherein the contact information includes at least one of the candidate's name, mailing address, email address, telephone number, and postal zip code.

9. The method according to claim 1, wherein generating an automated communication comprises generating the automated communication at a predetermined interval after the candidate completes the questionnaire.

10. The method according to claim 9, wherein the predetermined interval is approximately 24 hours or less.

11. The method according to claim 1, wherein if the candidate provides confirmation responsive to the automated communication, then the method further comprises contacting the candidate to complete screening for the clinical trial program.

12. The method according to claim 11, wherein contacting the candidate comprises contacting the candidate via at least one of telephone, email, or mail.

13. The method according to claim 11, wherein contacting the candidate comprises conducting a telephone interview with the candidate by a clinical trial representative.

14. The method according to claim 1, further comprising storing information obtained from the candidate.

15. The method according to claim 14, wherein storing information obtained from the candidate comprises. storing and managing the information in accordance with applicable laws and regulations.

16. The method according to claim 15, wherein the applicable laws and regulations comprise the Health Insurance Portability and Accountability Act and 21 C.F.R. Part 11.

17. The method according to claim 1, further comprising generating a report based on information obtained from the candidate.

18. The method according to claim 17, further comprising providing a clinical trial program representative access to the report via a secure Internet site or a secure Intranet site.

19. The method according to claim 18, wherein the report is individualized based on requests from the clinical trial program representative.

20. The method according to claim 17, further comprising providing the candidate access to the report via a secure Internet site or a secure Intranet site.

21. The method according to claim 20, wherein the report is individualized based on requests from the candidate.
